(19) United States
(12) Patent Application Publication
(10) Pub. No.: US 2004/0054161 A1
(43) Pub. Date: Mar. 18, 2004

(54) Stepwise solid-phase synthesis of peptide-oligonucleotide conjugates and supports therefor

(75) Inventors: Maxim Antopolskii, Kuopio (FI); Elena Azhayeva, Siilinjarvi (FI); Alex Azhayev, Siilinjarvi (FI)

Correspondence Address: BANNER & WITCOFF, 1001 G STREET N W, SUITE 1100, WASHINGTON, DC, 20001, US

(73) Assignee: Glen Research Corporation, Sterling, VA

(21) Appl. No.: 10/242,668

(22) Filed: Sep. 13, 2002

(51) Int. Cl. C07H 21/04; C08G 63/48; C08G 63/91

(52) U.S. Cl. 536/25.3; 525/54.2

(57) ABSTRACT

A support for peptide-oligonucleotide conjugate synthesis includes an article of manufacture according to the formula:

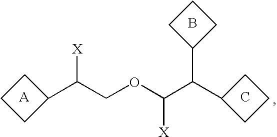

wherein: (a) substitutent A includes a polymeric base material and/or a silica base material; (b) one of substituents B and C includes an NHFmoc-containing group and/or a peptide-containing group; (c) one of substituents B and C includes an NHBoc-containing group, an O-DMTr-containing group, and/or an oligonucleotide-containing group; and (d) each X independently represents ═O, S, and/or NH. Suitable supports include those according to the formula:

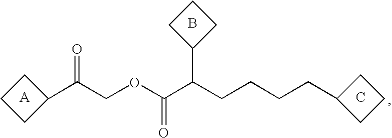

wherein substituents A, B, and C have the meanings described above. When used to synthesize a peptide-oligonucleotide conjugate, the resulting conjugate may have the formula:

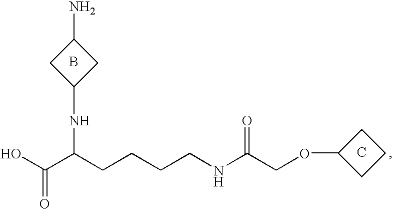

wherein substituent B includes a peptide and substituent C includes an oligonucleotide.

### FIELD OF THE INVENTION

This invention relates to solid supports for synthesizing peptide-oligonucleotide conjugates, as well as to methods for making such supports and to stepwise solid-phase synthesis methods for producing peptide-oligonucleotide conjugates.

### BACKGROUND OF THE INVENTION

Conjugates that contain both peptide and oligonucleotide moieties have many uses in molecular biology. For example, such conjugates may be used as non-radioactive labels, as PCR primers, and as “antiviral” and “antigene” inhibitors of gene expression. Additionally, covalent oligonucleotide-peptide conjugates have received considerable attention as anti-sense agents that exhibit potentially enhanced cellular uptake. This invention, in general, relates to supports and methods for making conjugates using the supports.

The preparation of such conjugates has been based either on post-synthetic conjugation of oligonucleotides and peptides, both having appropriate reactive groups, or on stepwise solid-phase assembly. All the post-synthetic conjugations described to date are associated with lengthy and troublesome procedures that include several intermediate purification steps. In contrast, stepwise solid-phase procedures to prepare oligonucleotide-peptide conjugates usually require only one purification step upon final deprotection. However, the choice of permanent protecting groups of nucleoside and amino-acid monomers, employed in the stepwise solid-phase synthesis, remains a critical factor.

No general methods for the stepwise synthesis of peptide-oligonucleotide phosphorothioate conjugates (“POPC”) on a single solid support have been reported. See, for example, C. Tung, “Preparation and Applications of Peptide-Oligonucleotide Conjugates,” Bioconjugate Chemistry, Vol. 11, No. 5 (2000), pp. 605-618, which article is entirely incorporated herein by reference. The conventional sequential assembly leads to a limited number of POPCs due to the incompatibility of the peptide and the oligonucleotide protecting groups. As a result, the use of various base-labile peptide side-chain protecting groups has been reported in the literature. See, for example, (a) J. Truffert, et al., “On-Line Solid Phase Synthesis of Oligonucleotide-Peptide Hydrids Using Silica Supports,” Tetrahedron Letters, Vol. 35, No. 15 (1994), pp. 2353-2356; (b) J. Truffert, et al., “Synthesis, Purification and Characterization of Two Peptide-Oligonucleotide Conjugates as Potential Artificial Nucleases,” Tetrahedron, Vol. 52, No. 8 (1996), pp. 3005-3016; (c) B. de la Torre, et al., “Stepwise Solid-Phase Synthesis of Oligonucleotide-Peptide Hybrids,” Tetrahedron Letters, Vol. 35, No. 17 (1994), pp. 2733-2736; (d) B. de la Torre, et al., “Synthesis and Binding Properties of Oligonucleotides Carrying Nuclear Localization Sequences,” Bioconjugate Chemistry, Vol. 10 (1999), pp. 1005-1012; (e) J. Robles, et al., “Towards Nucleopeptides Containing Any Trifunctional Amino Acid,” Tetrahedron, Vol. 55 (1999), pp. 13251-13264; and (f) M. Antopolsky, et al., “Stepwise Solid-Phase Synthesis of Peptide-Oligonucleotide Conjugates on New Solid Supports,” Helvetica Chimica Acta, Vol. 82 (1999), pp. 2130-2140. Each of these articles is entirely incorporated herein by reference.

Previously, a group including some of the present inventors described the synthesis of three conjugates containing 10 or 16-mer peptides, which incorporated two or three arginine residues. See M. Antopolsky, et al., “Stepwise Solid-Phase Synthesis of Peptide-Oligonucleotide Phosphorothioate Conjugates Employing Fmoc Peptide Chemistry,” Tetrahedron Letters, Vol. 41 (2000), pp. 9113-9117, which article is entirely incorporated herein by reference. The required peptides were assembled on the support by using common N_{α}-Fmoc amino-acids, but with Fmoc-Orn(Mtt)-OH as a precursor of -Arg-. The solid phases were then modified to offer doubly protected guanidine functions of arginines. Finally, oligonucleotides were assembled, and the POPCs were cleaved from the support and deprotected with aqueous ammonia.

The present invention relates to supports and procedures further developing general methods for the stepwise solid-phase synthesis of POPCs. These procedures, in at least some instances, also may use commercially available N_{α}-Fmoc amino-acids for peptide synthesis along with modifications after the peptide assembly and before the oligonucleotide assembly.

### SUMMARY OF THE INVENTION

The present invention relates to solid supports that may be used, for example, in peptide-oligonucleotide conjugate synthesis, methods of making the supports, and methods of using the supports in conjugate synthesis.

One aspect of this invention relates to supports according to the formula:

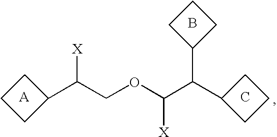

wherein: substitutent A includes at least one member selected from the group consisting of a polymeric base material and a silica base material; one of substituents B and C includes at least one member selected from the group consisting of an NHFmoc-containing group and a peptide-containing group; one of substituents B and C includes at least one member selected from the group consisting of an NHBoc-containing group, an O-DMTr-containing group, and an oligonucleotide-containing group; and X represents a member selected from the group consisting of ═O, S, and NH. In some examples of the invention, substituent A may include a controlled pore glass base material, such as a long chain alkylamino controlled pore glass base material (a “lcaa-CPG base material”).

In some supports according to the formula described above, substituent A is connected to a remaining portion of the support through a nitrogen atom or through an alkyl chain having 1 to 6 carbon atoms, substituent B optionally is connected to the remaining portion of the support through an alkyl chain having 1 to 6 carbon atoms, and substituent C optionally is connected to the remaining portion of the support through an alkyl chain having 1 to 6 carbon atoms.

Another aspect of this invention relates to supports according to the formula:

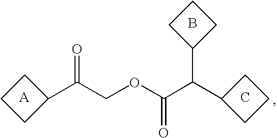

wherein substituents A, B, and C have the same definitions as those provided above.

Yet another aspect of this invention relates to supports according to the formula:

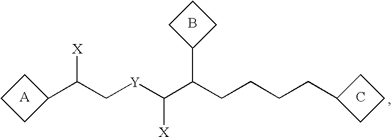

wherein substituents A, B, C, and X have the same definitions as provided above. Substituent Y in this formula represents a member selected from the group consisting of O and S.

Supports according to the invention also include those according to the following formula:

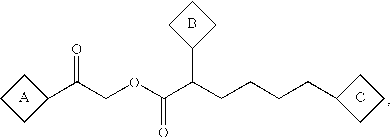

wherein substituents A, B, and C have the same definitions provided above. As illustrated in FIG. 1C, one exemplary support material according to this invention (support SS-1) is that represented by the following formula:

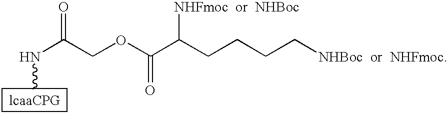

Another aspect of this invention relates to peptide-oligonucleotide conjugates according to the formula:

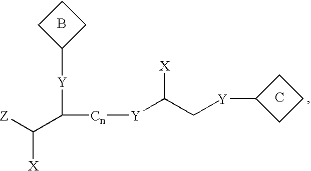

wherein substituent B includes a peptide or an oligonucleotide; substituent C includes an oligonucleotide or a peptide; n represents a number between 0 and 10; X represents a member selected from the group consisting of ═O, S, and NH; Y represents a member selected from the group consisting of O and NH; and Z represents a member selected from the group consisting of a hydroxy group, an alkyl group, and an aryl group.

Peptide-oligonucleotide conjugates according to the invention also include those according to the formula:

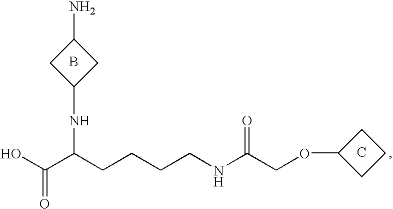

wherein substituent B includes a peptide and substituent C includes an oligonucleotide.

In some examples according to the invention, the definitions of substituents B and C in the above formulae are replaced with the following definitions: one of substituents B and C includes at least one member selected from the group consisting of an NHFmoc-containing group, a substituent group useful in peptide synthesis, a substituent group stable against oligonucleotide synthesis, and a peptide-containing group; and one of substituents B and C includes at least one member selected from the group consisting of an NHBoc-containing group, an O-DMTr-containing group, a substituent group stable against peptide synthesis, a substituent group useful in oligonucleotide synthesis, and an oligonucleotide-containing group.

This invention also relates to methods for making the supports for peptide-oligonucleotide conjugate synthesis, to methods for using the supports in peptide-oligonucleotide synthesis, and to intermediates prepared in making the supports and the conjugates according to the invention. Examples of the products and intermediates according to the invention include compositions 2, 3, SS-1, SS-2, SS-3, SS-4, SS-5, and the other materials illustrated in FIGS. 1A through 3B.

### BRIEF DESCRIPTION OF THE DRAWINGS

Various aspects of this invention are described in conjunction with the attached drawings, wherein:

FIGS. 1A to 1C illustrate an example of synthesis of an exemplary support for solid-phase peptide-oligonucleotide conjugate synthesis;

FIGS. 2A to 2D illustrate an example of a reaction scheme useful to attach a peptide to an exemplary support according to the invention and to prepare the supported peptide for conjugate synthesis; and

FIGS. 3A to 3B illustrate an example of a reaction scheme useful for producing a peptide-oligonucleotide conjugate.

### DETAILED DESCRIPTION OF THE INVENTION

As described above, aspects of this invention relate to solid supports for peptide-oligonucleotide synthesis, methods for making such supports, and methods for using the supports for solid-phase synthesis of peptide-oligonucleotide conjugates (e.g., peptide-oligonucleotide phosphorothioate conjugates). Conjugate synthesis procedures according to at least some examples of the invention may use commercially available N_{α}-Fmoc amino-acids for the peptide synthesis along with certain modifications after the peptide synthesis and before the oligonucleotide assembly.

The following definitions apply to this specification and the claims, unless another definition is provided.

The term “alkyl,” as used herein, refers to substituted or unsubstituted, straight or branched chain groups, preferably, having one to twelve, more preferably having one to six, and most preferably having from one to four carbon atoms. Illustrative examples of alkyl groups include, but are not limited to: methyl, trifluoromethyl, ethyl, propyl, isopropyl, butyl, isobutyl, t-butyl, pentyl, hexyl, and the like.

The term “aryl,” as used herein, refers to an aromatic, monovalent monocyclic, bicyclic, or tricyclic radical containing 6, 10, 14, or 18 carbon ring atoms, which may be unsubstituted or substituted, and to which may be fused one or more cycloalkyl groups, heterocycloalkyl groups, or heteroaryl groups, which themselves may be unsubstituted or substituted by one or more suitable substituents. Illustrative examples of aryl groups include, but are not limited to: phenyl, naphthyl, anthryl, phenanthryl, fluoren-2-yl, indan-5-yl, and the like.

Examples of possible substituents for alkyl and aryl groups include: mercapto, thioether, nitro (NO₂), amino, aryloxyl, halogen, hydroxyl, alkoxyl, and acyl, as well as aryl, cycloalkyl and saturated and partially saturated heterocycles.

The term “halogen” represents chlorine, fluorine, bromine, or iodine. The term “halo” represents chloro, fluoro, bromo or iodo.

The term “acyl,” as used herein, represents substituents of the general formula —C(═O)R, wherein R is hydrogen or an alkyl group. Illustrative examples of acyl groups include formyl, acetyl, chloroacetyl, dichloroacetyl, and propionyl.

The term “aroyl,” as used herein, represents an acyl group wherein the R substituent includes an aryl group. Illustrative examples of aroyl groups include phenoxyacetyl groups, halophenoxyacetyl groups (e.g., 4-chlorophenoxyacetyl), dihalophenoxyacetyl (e.g., 2,4-dichlorophenoxyacetyl), and the like.

The term “Boc” as used in this specification represents a t-butyloxycarbonyl group. The term “CPG” stands for controlled pore glass, and the term “lcaa-CPG” stands for long chain alkylamino controlled pore glass. The term “DMTr,” as used herein, represents a 4,4′-dimethoxytrityl group. The term “Fmoc,” as used herein, represents a 9-fluorenylmethyloxycarbonyl group.

Any solid support material suitable for use in oligonucleotide synthesis can be used with this invention. For example, the solid supports described in the literature identified in the “Background” section of this document can be used in conjunction with the invention. The solid supports can be beads, particles, sheets, dipsticks, rods, membranes, filters, fibers (e.g., optical or glass), or in any other suitable form. The material composition of the solid support materials may be any suitable materials, such as polymeric or silica based support materials. Specific examples, include plastic, nylon, glass, silica, metal, metal alloy, polyacrylamide, polyacrylate, polystyrene, cross-linked dextran, and combinations thereof. The term “silica base material” or “silica support material,” as used in the present specification and claims, includes glass materials, unless it is specifically indicated that glass materials are not included.

The term “oligonucleotide” as used in this document has its conventional meaning. The articles identified above illustrate the meaning of this term. One suitable definition, although not necessarily exclusive or exhaustive, is as follows: the term “oligonucleotide” is generic to polydeoxynucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, providing that the polymers contain nucleobases in a configuration that allows for base pairing and base stacking, such as is found in DNA and RNA. It will be appreciated that, as used herein, the terms “nucleoside” and “nucleotide” will include those moieties that contain not only the known purine and pyrimidine bases, but also modified purine and pyrimidine bases and other heterocyclic bases that have been modified (these moieties are sometimes referred to collectively as “purine and pyrimidine bases and analogs thereof”). Such modifications include methylated purines or pyrimidines, acylated purines or pyrimidines, and the like.

A wide variety of protecting groups can be employed in the supports and the synthesis procedures according to the invention, and examples of suitable protecting groups are described in the publications identified above. In general, protecting groups render chemical functionality inert to specific reaction conditions, and can be appended to and removed from such functionality in a molecule. Representative protecting groups are disclosed by Beaucage, S. L.; Uyer, R. P., “Advances in the Synthesis of Oligonucleotides by the Phosphoramidite Approach,” Tetrahedron, 1992, 48, 2223-2311, which article is entirely incorporated herein by reference. Illustrative protecting groups that can be removed under acidic or neutral conditions include trityl (Tr), dimethoxytrityl (DMTr), monomethoxytrityl, 9-phenylxanthen-9-yl (Pixyl), 9-(p-methoxyphenyl)xanthen-9-yl (Mox), and 4,4′,4″-tris-tert-butyltrityl (TTTr). Illustrative protecting groups that can be removed under neutral conditions are base-labile protecting groups, such as the acyl or aroyl groups identified above.

All inventive compounds that contain at least one chiral center may exist as single stereoisomers, racemates and/or mixtures of enantiomers and/or diastereomers. All such single stereoisomers, racemates and mixtures thereof are intended to be within the scope of the present invention.

In some examples of the invention, suitable solid supports are prepared containing Fmoc-protected amino-function for easy standard peptide chain elongation, Boc-protected amino function, which remains stable during the process of peptide synthesis but is easily removable for subsequent modification and, finally, base-labile bridges to solid matrix— long chain alkylamino controlled pore glass (lcaa-CPG).

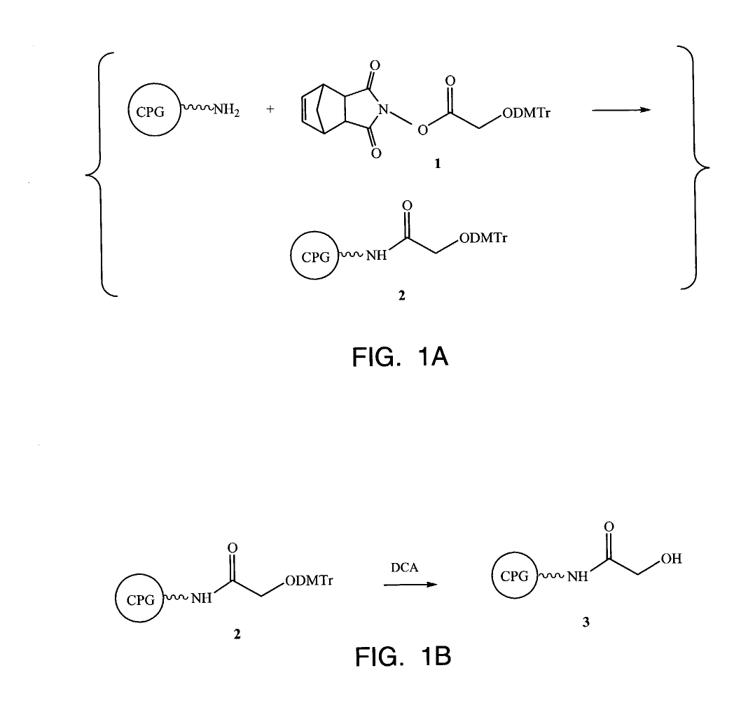

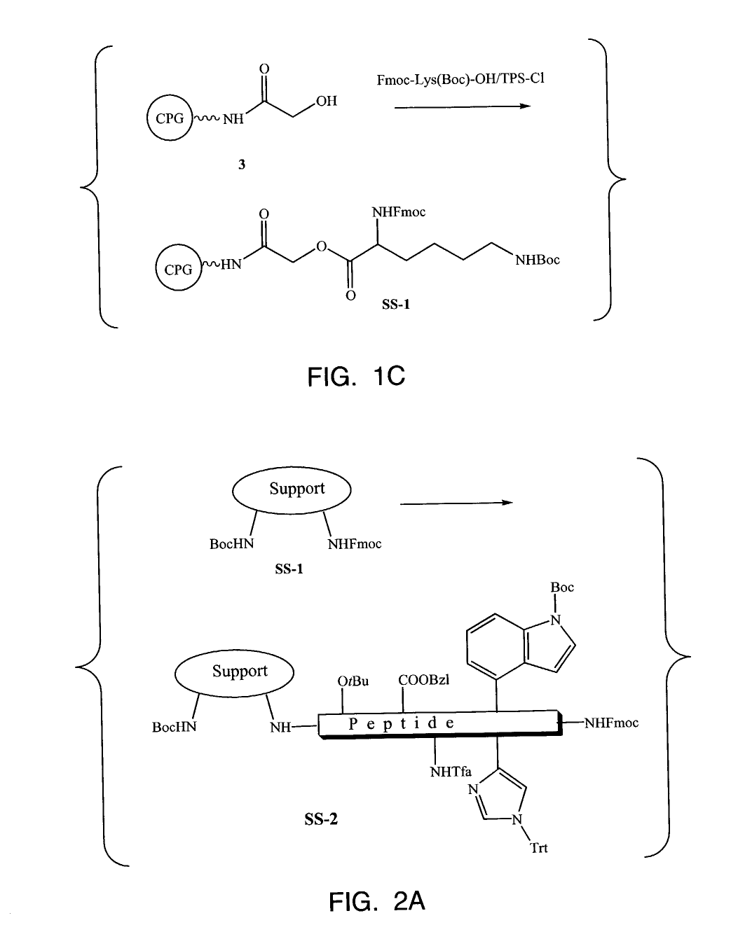

An example of the synthesis of solid support SS-1 is outlined in FIGS. 1A through 1C. While a detailed explanation of an example of the synthesis procedure is provided in the experimental section that follows, a brief explanation of the reaction schemes is provided below.

Initially, a long chain alkylamino controlled pore glass (lcaa-CPG) was derivatized by the N-hydroxy-5-norbomene-endo-2,3-dicarboxamide ester of 2-(4′,4″-dimethoxytriphenylmethyloxy) acetic acid (1) to give matrix 2 (FIG. 1A). After the removal of the DMTr-group (to give compound 3, see FIG. 1B), Fmoc-Lys(Boc)-OH was linked to the matrix 3 in the presence of 2,4,6-triisopropylbenzenesulfonyl chloride (TPS-Cl) to give solid support SS-1 (see FIG. 1C).

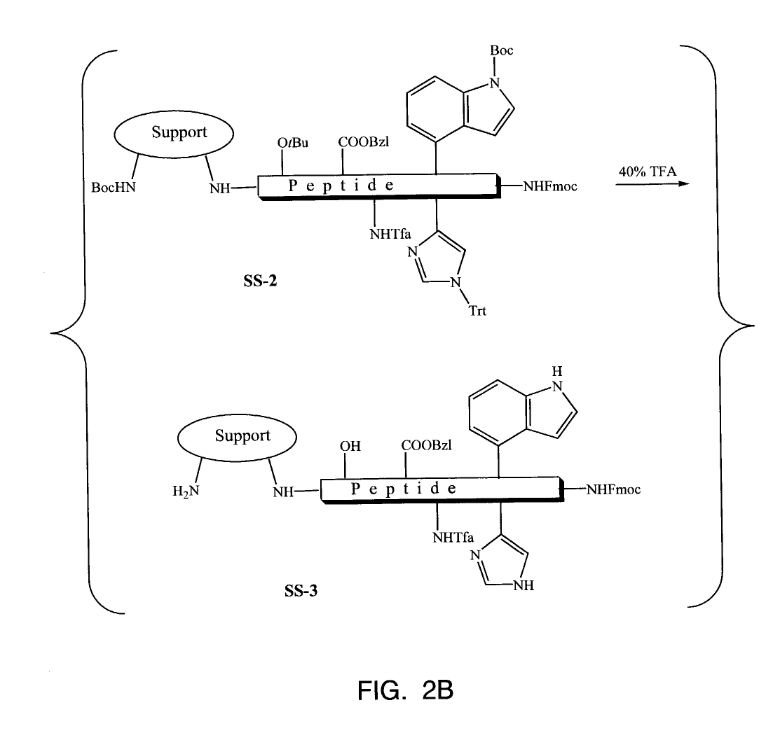

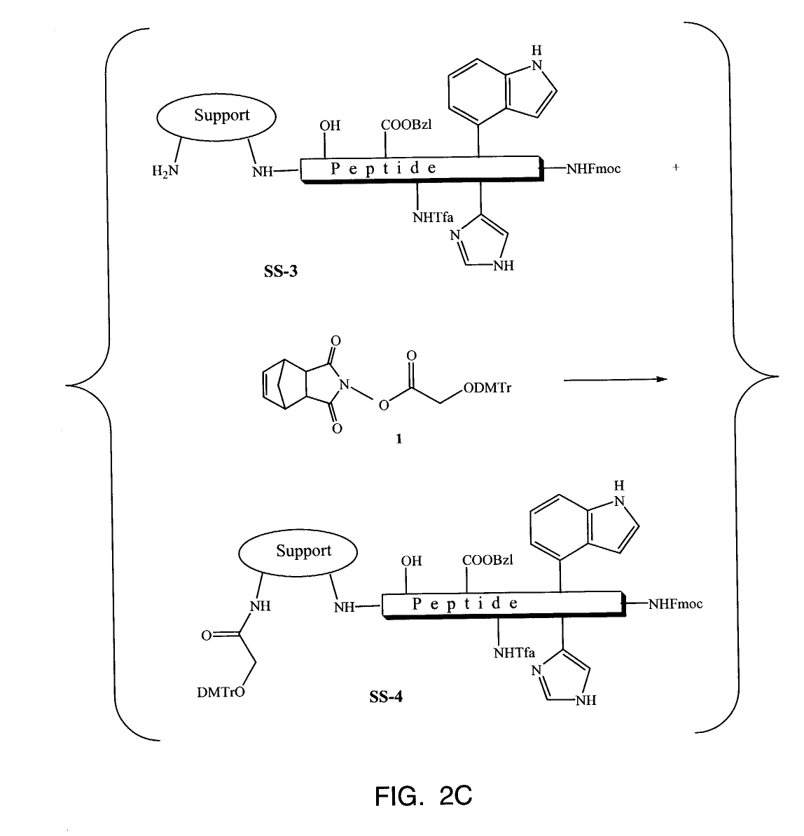

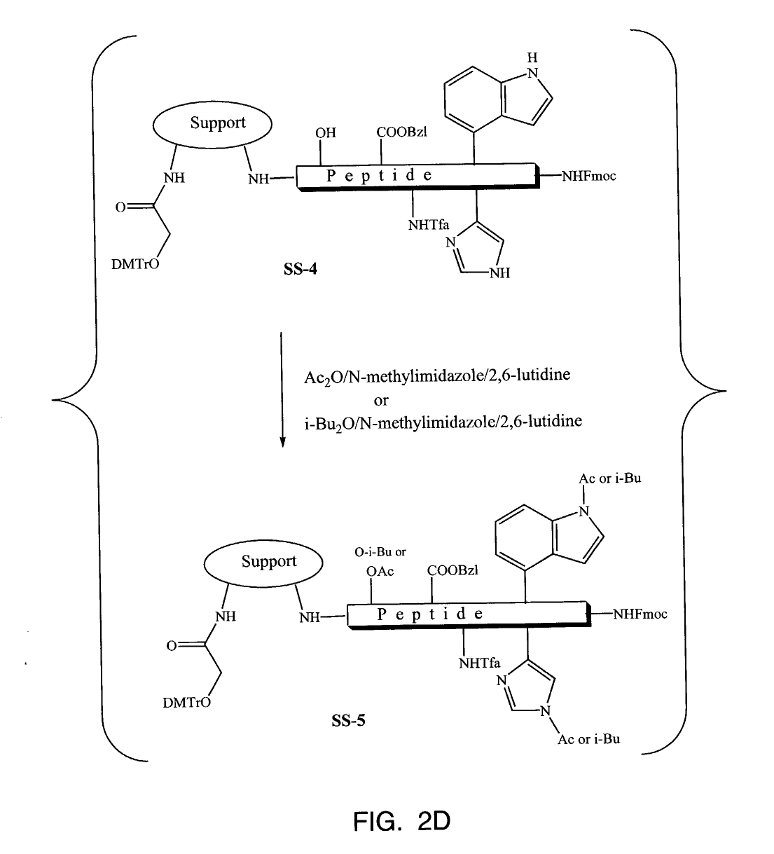

FIGS. 2A through 2D illustrate an example of the use of solid supports according to the invention (such as solid support SS-1) to produce a supported peptide and to make this supported peptide ready for conjugation with an oligonucleotide. Specifically, in this example, the solid support SS-5 is prepared incorporating the required peptide with base-labile side-chain protecting groups and a linker ready for oligonucleotide assembly. In this procedure, standard Fmoc-peptide chemistry and commercially available Na_{α}-Fmoc amino-acids may be employed to assemble peptides on the initial SS-1 matrix to give phases SS-2. Examples of N_{α}-Fmoc amino-acids include Fmoc-Met-OH, Fmoc-Tyr(tBu)-OH, Fmoc-Ile-OH, Fmoc-Glu(OBzl)-OH, Fmoc-Ala-OH, Fmoc-Leu-OH, Fmoc-Asp(OBzl)-OH, Fmoc-Lys(OTfa)-OH, Fmoc-Cys(Acm)-OH, Fmoc-His(Trt)-OH, Fmoc-Ser(tBu)-OH, and Fmoc-Thr(tBu)-OH. Other suitable amino acids also may be used without departing from the invention.

Examples of peptides that were assembled as part of the synthesis procedure illustrated in FIGS. 2A to 2D include H-Me0t-Tyr-Ile-Glu-Ala-Leu-Asp-Lys-Tyr-Ala-Cys(Acm)-OH (P-1) and H-Met-His-Ile-Glu-Ser-Leu-Asp-Ser-Tyr-Thr-Cys(Acm)-OH (P-2), although any suitable peptide may be supported without departing from the invention. After the peptide assembly, all acid-labile protecting groups were removed from the peptides attached to the solid phase support, which resulted in the SS-3 supports (see FIG. 2B).

The primary amino-group of the SS-3 supports was then selectively derivatized with compound 1 (see FIG. 1A) to give supports SS-4, incorporating a DMTr-oxy function (reaction shown in FIG. 2C). The remaining unprotected side-chain functional groups were finally acylated to give rise to the SS-5 supports (see FIG. 2D), ready for oligonucleotide assembly. In the examples using the two peptides described above, both resulting SS-5 matrixes contained about 32 μmols of DMTr-groups per gram of CPG.

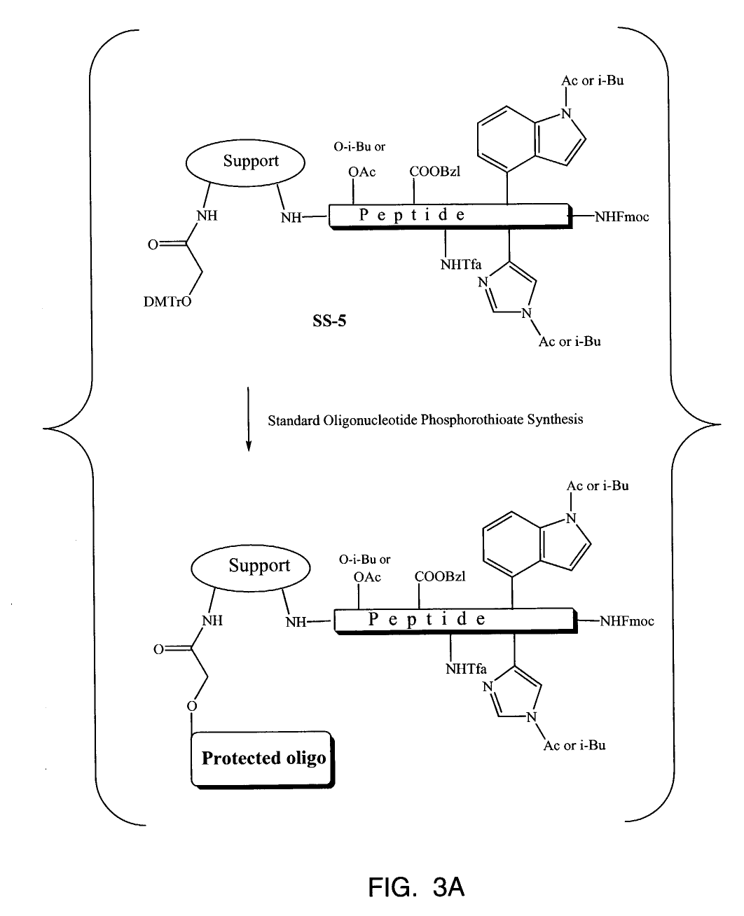

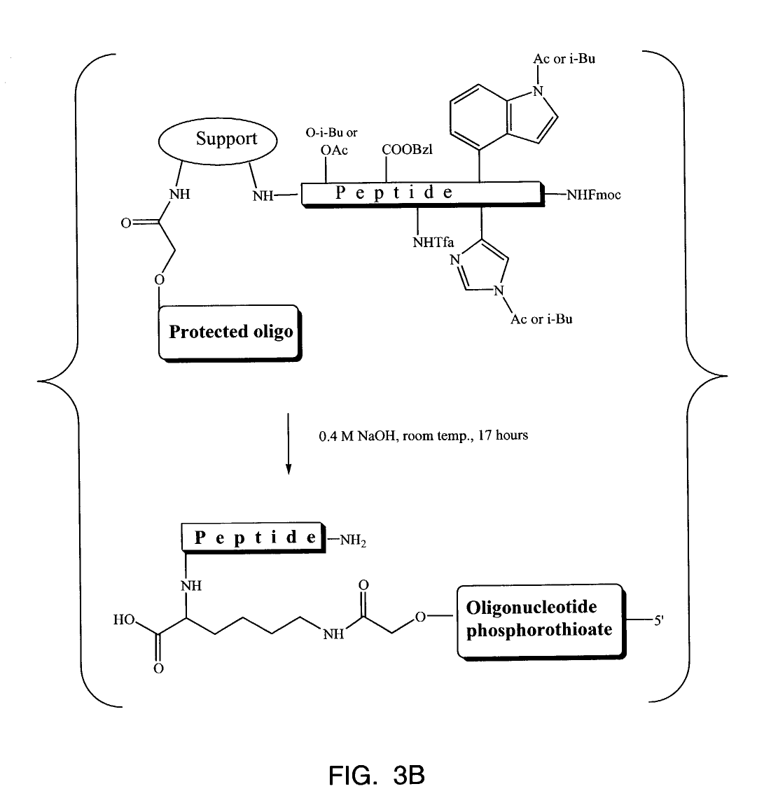

Two oligonucleotide phosphorothioates (5′-TGGCGTCTTCCATTT-3′ (O-1) and 5′-TATGATCTGTCACAGCTTGA-3′ (O-2) were assembled on the SS-5 supports using standard protocols. See FIG. 3A. Protected peptide-oligonucleotide phosphorothioate conjugates were cleaved from the support and deprotected to give rise to the crude conjugates, O-1-P-1, O-2-P-1, and O-2-P-2 (see FIG. 3B). After purification by ion exchange HPLC and desalting of these samples (e.g., by the process described in Antopolsky, et al., Helvetica Chimica Acta, supra), the purity of the POPCs was higher than 95%, as determined by RP HPLC. Typically 20-30 AU of pure POPCs were obtained when starting from 1 μmol of SS-5 solid supports.

As illustrated in FIGS. 2D through 3B, one or more of the Ac substituent groups on the peptide may be replaced with iso-butyl (“i-Bu”) substituent groups.

Details of examples of reaction conditions and procedures used in syntheses according to the invention follow in the Experimental Section.

I. Experimental Section

Various examples of syntheses and reaction schemes for making and using supports according to the invention are described in detail in the examples that follow. These examples should be construed as illustrating the invention, not as limiting it.

A. General Background Information Relating to the Examples

Various reactants and starting materials for the reaction schemes described below may be obtained from commercial sources. For example, 4,4′-dimethoxytrityl chloride may be obtained from ChemGenes, and long chain alkylamino controlled pore glass (“lcaaCPG”) may be obtained from various sources, such as Sigma and Glen Research. Reagents (solvents, activators, etc.) for oligonucleotide synthesis may be obtained, for example, from Glen Research. Standard oligonucleotide synthesis may be used according to the invention, for example, like that described in T. Atkinson, et al., Oligonucleotide Synthesis: A Practical Approach, Chapter 3, “Solid-Phase Synthesis of Oligodeoxyribonucleotides by the Phosphite-Triester Method,” 1984, M. Gait, ed., IRL Press, Oxford, pp. 35-81, which excerpt is entirely incorporated herein by reference.

Unless otherwise noted, the following equipment and test conditions were used in the experiments described below. Column flash chromatography was performed on Silica gel 60 (available from Merck). NMR spectra were recorded on a Brucker 500 spectrometer with tetramethylsilane as an internal standard (the following abbreviations are used in describing the NMR spectra: s=singlet; d=doublet; dd=doublet of doublet; m=multiplet; br.s=broad signal). All compounds bearing free hydroxy- and amino-groups were co-evaporated with CD₃OD before measurements were taken.

Oligonucleotides were analyzed by ion exchange HPLC (column: Dionex DNAPac PA-100, 4×250 mm, buffer A: 0.1 M NaAc in 20% MeCN, pH8, buffer B: 0.1 M NaAc and 0.4 M NaClO₄ in 20% MeCN, pH8; flow rate 1 ml min⁻¹; a linear gradient from 0 to 15% B in 20 minutes for oligo-T₆ and modified oligo-T₆ oligonucleotides and from 5 to 45% B in 30 minutes for longer oligomers). Phosphorothioate oligonucleotides were analyzed by ion exchange HPLC (column: Poly LP PolyWax 4.6×100 mm, 5 μm, 300 Å, buffer A: 0.05 M KH₂PO₄ in 50% formamide, pH 6.7, buffer B: 0.05 M KH₂PO₄ and 1.5 M NaBr in 50% formamide, pH 6.7; flow rate 1 ml min⁻¹; a linear gradient from 5 to 50% B in 30 min).

In the oligodeoxyribonucleotide synthesis described below, the protected oligonucleotides were assembled on an Applied Biosystems 392 DNA Synthesizer using phosphoroamidite chemistry and recommended DMTr-off protocols for 40 nmol, 0.2 μmol, and 1 μmol scale.

B. Preparation of Solid Support

One aspect of this invention relates to solid support materials useful in preparing peptide-oligonucleotide conjugates. FIGS. 1A through 1C illustrate an example of a reaction scheme useful for synthesizing one example of such a solid support material. The procedures illustrated in these drawings are described in more detail below.

1. Preparation of Compound 1

Compound 1, one of the starting materials for the reaction scheme illustrated in FIG. 1A, may be prepared as follows.

Glycolic acid (22 mmol, 1.67 g) was dissolved in 100 ml of dry pyridine. 4,4′-Dimethoxytrityl chloride (DMTr-Cl) (20 mmol, 6.76 g), dissolved in 50 ml of dry tetrahydrofuran (THF), was added, and the mixture was stirred at room temperature overnight. The reaction was quenched by the addition of ice. The resulting mixture was concentrated, dissolved in ethyl acetate, and extracted with water. The organic layer was dried, concentrated, and finally purified by flash chromatography on silica gel. Concentration in vacuo gave 5.88 g (78%) of a pale yellow oil. This oil (11 mmol, 4.4 g) and N-hydroxy-5-norbornene-endo-2,3-dicarboxamide (1.97 g, 11 mmol) were dissolved in 50 ml of dry acetonitrile, the solution was cooled down to −20° C., and a solution of N,N′-dicyclohexylcarbodiimide (11 mmol, 2.31 g) in 10 ml of acetonitrile was added. The reaction mixture was left at +4° C. overnight. Solids were filtered off and washed with cold acetonitrile. The filtrate and washings were combined and evaporated to dryness. Crystallization of the residue from 2-propanol gave 3.8 g (65%) of Compound 1 as white crystals.

The resulting Compound 1 had the following characteristics: m.p. 52-54° C.; ¹H NMR (CD₃CN): δ7.43-6.85 (m, 13H, arom.); 6.11 (s, 2H, CHₐ═CH_{b}); 4.04 (s, 2H, CHₐH_{b}CO); 3.75 (s, 6H, 2×CH₃OC₆H₄); 3.34 (s, 2H, 2×COCH); 3.32 (s, 2H, 2×CH—CH═CH); 1.66 (d, 1H, J=8.85, (CH—CHₐH_{b}—CH); 1.51 (d, 1H, J=8.85, (CH—CHₐH_{b}—CH). Found: C, 71.49; H, 5.51; N, 2.32. Calcd. For C₃₂H₂₉NO₇: C, 71.23; H, 5.42; N, 2.60%.

2. Preparation of Compound 2

A long chain alkylamino controlled pore glass (lcaa-CPG) was derivatized by the N-hydroxy-5-norbornene-endo-2,3-dicarboxamide ester of 2-(4′,4″-dimethoxytriphenylmethyloxy) acetic acid (Compound 1) to give Compound 2, as illustrated in FIG. 1A. The procedure was as follows: 2 g of lcaa-CPG was suspended in 7 ml of pyridine. 0.54 g (1 mmol) of 4,4′-Dimethoxytritylglycolic acid N-Oxy-5-norbomene-endo-2,3-dicarboximide ether and 0.15 g (1 mmol) of N-Hydroxybenzotriazole were added, and the reaction mixture was left shaking overnight at room temperature. The support was filtered, washed with pyridine (2×10 ml), tetrahydrofurane (2×10 ml), acetonitrile (2×10 ml), and ether (2×10 ml), and dried. The resulting solid support was treated with Ac₂O: Lutidine: THF=1 ml: 1 ml: 5.5 ml and N-methyl-imidazole: THF=1.6 ml: 5.9 ml for 2 hours. The support 2 was washed with tetrahydrofurane (2×10 ml), acetonitrile (2×10 ml), and ether (2×10 ml) and dried.

The support 2 contained 33 μmol of DMTr-groups per gram of CPG.

3. Preparation of Compound 3

In the next step, as illustrated in FIG. 1B, the DMTr groups of Compound 2 were removed by reaction with 2% dichloroacetic acid (DCA), by weight, in dichloromethane. The reaction procedure was as follows: Support 2 (2 g) was treated with 20 ml of 2% dichloroacetic acid in dichloromethane for 5 minutes at room temperature, filtered, washed with dichloromethane (2×10 ml), tetrahydrofurane (2×10 ml), acetonitrile (2×10 ml), and ether (2×10 ml), and dried to give Support 3.

4. Preparation of SS-1

Solid support SS-1 was prepared, as illustrated in FIG. 1C, by linking Fmoc-Lys(Boc)-OH to Support 3 in the presence of 2,4,6-triisopropylbenzenesulfonyl chloride (TPS-Cl). The procedure was as follows: 1.4 gram (3 mmol) of Fmoc-Lys(Boc)-OH and 0.25 ml of N-methyl-imidazole were dissolved in 7 ml of dry pyridine. 2 grams of Support 3 were suspended in this solution, and 0.87 grams (2.9 mmol) of 2,4,6-triisopropylbenzenesulfonyl chloride were added. The reaction mixture was left shaking overnight at room temperature. The support was filtered off, washed with pyridine (2×10 ml), tetrahydrofurane (2×10 ml), acetonitrile (2×10 ml), and ether (2×10 ml) and dried to provide solid support SS-1.

The resulting solid support SS-1 contained 35 μmols of Fmoc-groups per gram of the CPG.

C. Supporting a Peptide on the Solid Support

FIGS. 2A through 2D illustrate exemplary steps involved in supporting a peptide on an exemplary solid support according to the invention and preparing this support for an oligonucleotide conjugation reaction.

1. Preparation of SS-2

As illustrated in FIG. 2A, in a first step of this exemplary procedure, a peptide first is assembled onto the initial solid support SS-1 to give supported peptide SS-2. Standard Fmoc-peptide chemistry and commercially available N_{α}-Fmoc amino-acids were employed. See, for example, the Novabiochem 2002/3 Catalog, Synthesis Note Index, page I-29, 3.1-3.5 and pages 1-61, which document is entirely incorporated herein by reference.

Any suitable N_{α}-Fmoc amino-acids may be used without departing from the invention. For example, suitable N_{α}-Fmoc amino-acids may include: Fmoc-Met-OH, Fmoc-Tyr(tBu)-OH, Fmoc-Ile-OH, Fmoc-Glu(OBzl)-OH, Fmoc-Ala-OH, Fmoc-Leu-OH, Fmoc-Asp(OBzl)-OH, Fmoc-Lys(OTfa)-OH, Fmoc-Cys(Acm)-OH, Fmoc-His(Trt)-OH, Fmoc-Ser(tBu)-OH, and Fmoc-Thr(tBu)-OH. Also, any suitable peptide may be added to the support without departing from the invention. Two different peptides that were used in this synthesis procedure include: H-Met-Tyr-Ile-Glu-Ala-Leu-Asp-Lys-Tyr-Ala-Cys(Acm)-OH (P-1), and H-Met-His-Ile-Glu-Ser-Leu-Asp-Ser-Tyr-Thr-Cys(Acm)-OH (P-2).

2. Preparation of SS-3

The supported peptides SS-2 were further reacted with 40% trifluoroacetic acid (TFA) to remove the acid-labile protecting groups, including those attached to the solid phase, to thereby give supported peptides SS-3, as illustrated in FIG. 2B. The procedure was as follows: 300 mg of peptide bound support SS-2 was treated with 40% TFA in dichloromethane (DCM), containing 1% of 1,2-ethanedithiol for 20 minutes at room temperature. The resulting support SS-3 was filtered, washed with dichloromethane (2×10 ml), 1% triethylamine in dichloromethane (2×10 ml), and ether (2×10 ml), and dried.

3. Preparation of SS-4

The primary amino group of SS-3 then was selectively derivatized with Compound 1 to give SS-4, as illustrated in FIG. 2C. The procedure was as follows: Dry support SS-3 was suspended in 5 ml of tetrahydrofurane. 500 mg of Compound 1 and 153 mg of 1-hydroxybenzotriazole (HOBT) were added, and the reaction mixture was left overnight shaking at room temperature. The resulting support SS-4 was filtered off, washed with tetrahydrofurane (2×10 ml), acetonitrile (2×10 ml), and ether (2×10 ml), and dried.

As evident from the structure illustrated in FIG. 2C, SS-4 includes a DMTr-oxy functional group.

4. Preparation of SS-5

The remaining unprotected side-chain functional groups of support SS-4 were acylated to thereby produce the SS-5 supports, as illustrated in FIG. 2D, using Ac₂O/N-methylimidazole/2,6-lutidine or isobutyric anhydride/N-methylimidazole/2,6-lutidine. The procedure was as follows: The support SS-4 was acylated with either a mixture of acetic anhydride-2,6-lutidine-tetrahydrofurane (5 ml: 0.5 ml: 3 ml) and N-methyl-imidazole-tetrahydrofurane (0.8 ml: 3 ml ) for 2 hours at room temperature, or with a mixture of isobutyric anhydride-2,6-lutidine-tetrahydrofurane (5 ml: 0.5 ml: 3 ml) and N-methyl-imidazole-tetrahydrofurane (0.8 ml: 3 ml) overnight at room temperature. The resulting support SS-5 was filtered, washed with tetrahydrofurane (2×10 ml), acetonitrile (2×10 ml), and ether (2×10 ml), and dried.

The supported peptides SS-5 were ready for oligonucleotide conjugation. Both SS-5 matrices (i.e., those made with each of the peptides P-1 and P-2, as described above) contained 32 μmols of DMTr-groups per gram of the CPG.

D. Preparing the Conjugate

FIGS. 3A through 3C illustrate exemplary steps involved in preparing a peptide-oligonucleotide conjugate using an exemplary solid support according to the invention. In these examples, the following two oligonucleotides were assembled on the SS-5 supports using standard oligonucleotide phosphorothioate synthesis protocols: (a) “O-1” is 5′-TGGCGTCTTCCATTT-3′ and “O-2” is 5′-TATGATCTGTCACAGCTTGA-3′. First, as illustrated in FIG. 3A, a protected oligonucleotide is assembled on support SS-5 using standard oligonucleotide phosphorothioate synthesis conditions, to produce the product illustrated in the figure. Then, as illustrated in FIG. 3B, protected peptide-oligonucleotide phosphorothioate conjugates were cleaved from the support and deprotected with 0.4 M sodium hydroxide at room temperature for 17 hours to provide the crude conjugates O-1/P-1, O-2/P-2, and O-2/P-1.

The standard oligonucleotide phosphorothioate synthesis protocols used in this reaction step may be like those described in R. P. Iyer, W. Egan, J. B. Regan, and S. L. Beaucage, “3H-1,2-Benzodithiole-3-one 1,1-Dioxide as an Improved Sulfurizing Reagent in the Solid-Phase Synthesis of Oligodeoxyribonucleoside Phosphorothioates,” Journal of the American Chemical Society, 1990, Vol. 112, pp. 1253-1254. This document is entirely incorporated herein by reference.

Conjugates according to this invention can be used in any suitable manner, including in procedures and methods conventional and well known to those skilled in the art.

M. Antopolsky, et al., “Towards a General Method for the Stepwise Solid-Phase Synthesis of Peptide-Oligonucleotide Conjugates,” Tetrahedron Letters, Vol. 43 (2002), pp. 527-530 is hereby incorporated by reference.

While the invention has been described in terms of specific examples and embodiments, those skilled in the art will appreciate that there are numerous variations and permutations of the above described compositions and methods that fall within the spirit and scope of the invention as set forth in the appended claims.

We claim:

1. A support according to the formula: 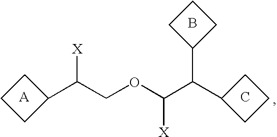
wherein:
substitutent A includes at least one member selected from the group consisting of a polymeric base material and a silica base material;
one of substituents B and C includes at least one member selected from the group consisting of an NHFmoc-containing group and a peptide-containing group;
one of substituents B and C includes at least one member selected from the group consisting of an NHBoc-containing group, an O-DMTr-containing group, and an oligonucleotide-containing group; and
each X independently represents a member selected from the group consisting of ═O, S, and NH.

2. A support according to claim 1, wherein substituent A includes a long chain alkylamino controlled pore glass base material.

3. A support according to claim 1, wherein substituent A is connected to a remaining portion of the support through a nitrogen atom or through an alkyl chain having 1 to 6 carbon atoms, wherein substituent B optionally is connected to the remaining portion of the support through an alkyl chain having 1 to 6 carbon atoms, and wherein substitutent C optionally is connected to the remaining portion of the support through an alkyl chain having 1 to 6 carbon atoms.

4. A support according to the formula: 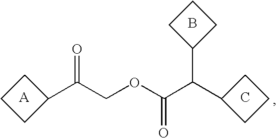
wherein:
substitutent A includes at least one member selected from the group consisting of a polymeric base material and a silica base material;
one of substituents B and C includes at least one member selected from the group consisting of an NHFmoc-containing group and a peptide-containing group; and
one of substituents B and C includes at least one member selected from the group consisting of an NHBoc-containing group, an O-DMTr-containing group, and an oligonucleotide-containing group.

5. A support according to claim 4, wherein substituent A is connected to a remaining portion of the support through a nitrogen atom or through an alkyl chain having 1 to 6 carbon atoms, wherein substituent B optionally is connected to the remaining portion of the support through an alkyl chain having 1 to 6 carbon atoms, and wherein substitutent C optionally is connected to the remaining portion of the support through an alkyl chain having 1 to 6 carbon atoms.

6. A support according to the formula: 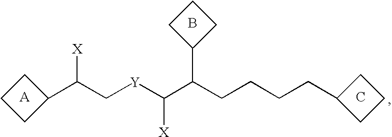
wherein:
substitutent A includes at least one member selected from the group consisting of a polymeric base material and a silica base material;
one of substituents B and C includes at least one member selected from the group consisting of an NHFmoc-containing group and a peptide-containing group;
one of substituents B and C includes at least one member selected from the group consisting of an NHBoc-containing group, an O-DMTr-containing group, and an oligonucleotide-containing group;
each X independently represents a member selected from the group consisting of ═O, S, and NH; and
Y represents a member selected from the group consisting of O and S.

7. A support according to the formula: 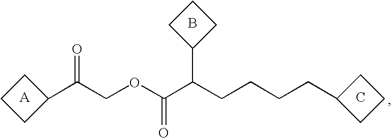
wherein:
substitutent A includes at least one member selected from the group consisting of a polymeric base material and a silica base material;
one of substituents B and C includes at least one member selected from the group consisting of an NHFmoc-containing group and a peptide-containing group; and
one of substituents B and C includes at least one member selected from the group consisting of an NHBoc-containing group, an O-DMTr-containing group, and an oligonucleotide-containing group.

8. A support according to the formula: 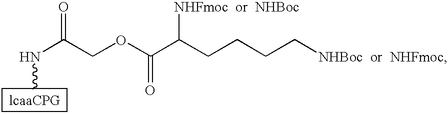
wherein lcaaCPG represents a long chain alkylamino controlled pore glass base material, and
wherein the support includes at least one NHFmoc group and at least one NHBoc group.

9. A peptide-oligonucleotide conjugate according to the formula: 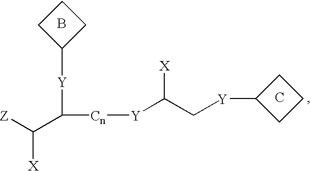
wherein:
substituent B includes a peptide or an oligonucleotide;
substituent C includes an oligonucleotide or a peptide;
n represents a number between 0 and 10;
each X independently represents a member selected from the group consisting of ═O, S, and NH;
each Y independently represents a member selected from the group consisting of O, S, and NH; and
Z represents a member selected from the group consisting of a hydroxy group, an alkyl group, and an aryl group.

10. A peptide-oligonucleotide conjugate according to the formula: 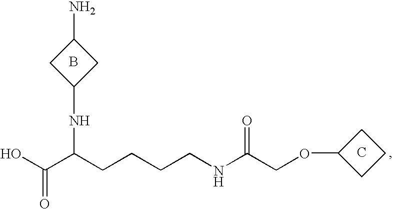
wherein:
substituent B includes a peptide; and
substituent C includes an oligonucleotide.

11. A method for preparing a peptide-oligonucleotide conjugate comprising:
providing a support according to the formula: 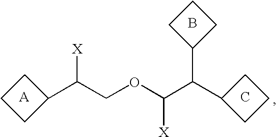
wherein:
substitutent A includes at least one member selected from the group consisting of a polymeric base material and a silica base material,
one of substituents B and C includes an NHFmoc-containing group,
one of substituents B and C includes a member selected from the group consisting of an NHBoc-containing group and an O-DMTr-containing group, and
each X independently represents a member selected from the group consisting of ═O, S, and NH;
synthesizing a peptide-containing group at the NHFmoc-containing substituent group; and
synthesizing an oligonucleotide-containing group at the NHBoc- or O-DMTr-containing substituent group.

12. A method according to claim 11, wherein substituent A includes a long chain alkylamino controlled pore glass base material.

13. A method according to claim 11, wherein substituent A is connected to a remaining portion of the support through a nitrogen atom or through an alkyl chain having 1 to 6 carbon atoms, wherein substituent B optionally is connected to the remaining portion of the support through an alkyl chain having 1 to 6 carbon atoms, and wherein substitutent C optionally is connected to the remaining portion of the support through an alkyl chain having 1 to 6 carbon atoms.

14. The method according to claim 11, further comprising: removing the support after the peptide synthesis and oligonucleotide synthesis steps.

15. A method for preparing a peptide-oligonucleotide conjugate, comprising:
providing a support according to the formula: 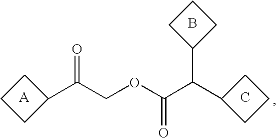
wherein:
substitutent A includes at least one member selected from the group consisting of a polymeric base material and a silica base material,
one of substituents B and C includes an NHFmoc-containing group, and
one of substituents B and C includes a member selected from the group consisting of an NHBoc-containing group and an O-DMTr-containing group;
synthesizing a peptide-containing group at the NHFmoc-containing substituent group; and
synthesizing an oligonucleotide-containing group at the NHBoc- or O-DMTr-containing substituent group.

16. A method according to claim 15, wherein substituent A is connected to a remaining portion of the support through a nitrogen atom or through an alkyl chain having 1 to 6 carbon atoms, wherein substituent B optionally is connected to the remaining portion of the support through an alkyl chain having 1 to 6 carbon atoms, and wherein substitutent C optionally is connected to the remaining portion of the support through an alkyl chain having 1 to 6 carbon atoms.

17. The method according to claim 15, further comprising: removing the support after the peptide synthesis and oligonucleotide synthesis steps.

18. A method for preparing a peptide-oligonucleotide conjugate, comprising:
providing a support according to the formula: 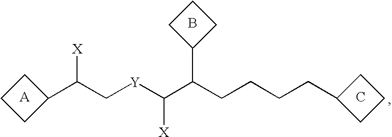
wherein:
substitutent A includes at least one member selected from the group consisting of a polymeric base material and a silica base material,
one of substituents B and C includes an NHFmoc-containing group,
one of substituents B and C includes at least one member selected from the group consisting of an NHBoc-containing group and an O-DMTr-containing group,
each X independently represents a member selected from the group consisting of ═O, S, and NH, and
Y represents a member selected from the group consisting of O and S;
synthesizing a peptide-containing group at the NHFmoc-containing substituent group; and
synthesizing an oligonucleotide-containing group at the NHBoc- or O-DMTr-containing substituent group.

19. The method according to claim 18, further comprising: removing the support after the peptide synthesis and oligonucleotide synthesis steps.

20. A method for preparing a peptide-oligonucleotide conjugate, comprising:
providing a support according to the formula: 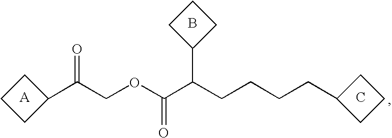
wherein:
substitutent A includes at least one member selected from the group consisting of a polymeric base material and a silica base material,
one of substituents B and C includes an NHFmoc-containing group, and
one of substituents B and C includes at least one member selected from the group consisting of an NHBoc-containing group and an O-DMTr-containing group;
synthesizing a peptide-containing group at the NHFmoc-containing substituent group; and
synthesizing an oligonucleotide-containing group at the NHBoc- or O-DMTr-containing substituent group.

21. The method according to claim 20, further comprising: removing the support after the peptide synthesis and oligonucleotide synthesis steps.

22. A method for preparing a peptide-oligonucleotide conjugate, comprising:
providing a support according to the formula: 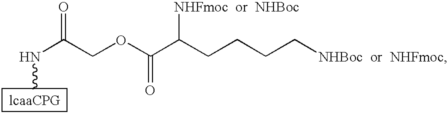
wherein lcaaCPG represents a long chain alkylamino controlled pore glass base material, and
wherein the support includes at least one NHFmoc group and at least one NHBoc group;
synthesizing a peptide-containing group at the NHFmoc-containing group; and
synthesizing an oligonucleotide-containing group at the NHBoc-containing group.

23. The method according to claim 22, further comprising: removing the support after the peptide synthesis and oligonucleotide synthesis steps.
